(19) United States
(12) Patent Application Publication
(10) Pub. No.: US 2005/0265954 A1
(43) Pub. Date: Dec. 1, 2005

(54) Novel association, a method of its preparation and its uses

(75) Inventors: Peter Petrov, Minsk (BY); Tatsiana Trukhachova, Minsk (BY); Henadz Isakau, Minsk (BY); Tomasz Stawinski, Dziechciniec (PL); Maciej Adamkiewicz, Lomianki (PL); Slawomir Mirek, Warsaw (PL)

Correspondence Address: DARBY & DARBY P.C., P. O. BOX 5257, NEW YORK, NY, 10150-5257, US

(73) Assignee: ADAMED, Sp. z.o.o., Czosnow K/Warszawy (PL)

(21) Appl. No.: 11/102,139

(22) Filed: Apr. 7, 2005

(30) Foreign Application Priority Data
May 25, 2004 (PL) P-368213

(51) Int. Cl. A61K 31/785; A61K 31/765; A61K 31/409

(52) U.S. Cl. 424/78.3; 514/410; 604/20

(57) ABSTRACT

The invention provides a novel association of polyvinylpyrrolidone with chlorin p6 and/or its salt with a pharmaceutically acceptable counter-ion and a method of preparation thereof. Preferably, the weight ratio of chlorin p6 and/or its salt with a pharmaceutically acceptable counter-ion to polyvinylpyrrolidone is contained within the range from about 1:1 to about 1:200. The association can be used as a photosensitizer in photodynamic therapy and/or diagnostics or for photochemical internalization of molecules as well as a photosensitizer in cosmetology. The invention provides also a pharmaceutical composition containing the novel association, a method of treating and diagnosing diseases by a dynamic phototherapy and a method of performing cosmetic procedures with the use of the novel association as a photosensitizer.

This invention provides a novel association of chlorin p6 useful as a photosensitizer in particular in photodynamic therapy and diagnostics of various conditions and diseases including neoplastic diseases as well as in cosmetology. More specifically, the invention provides a novel molecular association of chlorin p6 with polyvinylpyrrolidone (PVP), a method of manufacturing thereof as well as its uses.

The photodynamic therapy consists in systemic or topical administration of a photosensitizer that selectively accumulates in the target tissue of a human or an animal, e.g. in the pathologically changed or damaged tissue. Following exposure of such tissue to light of a defined wavelength in the visible region of spectrum, the photosensitizer produces cytotoxic species that destroy tissues, e.g. the pathologically changed or damaged tissues. Destruction of cells by cytotoxic species, via necrosis or apoptosis leads to breaking down the functions of the tissue and its destruction. Simultaneously, the irradiation induces fluorescence of the photosensitizer that is a sensitive diagnostic tool suitable for detecting the regions of the body which are abnormal in terms of their structural and functional condition or where intense biological processes occur, including formation of benign and malignant neoplasms, inflammatory processes, etc.

The chlorophyll derivatives, chlorin p6 (13-carboxy-17-[2-carboxyethyl]-15-carboxy-17,18-trans-dihydro-3-vinyl-8-ethyl-2,7,12,18-tetramethylporphyrin) and its anhydride analog, purpurin-18, that are disclosed in Photochemistry and Photobiology, 48:579 (1988) as well as in Ross. Fotodinam. Zhournal, 5, 1995, are strong photosensitizers and promote cell killing by low-intensity light.

A chlorin-based photosensitizer, comprising a mixture of chlorin e6, purpurin 5 and chlorin p6 in a form of an alkali metal salt, a method of manufacturing thereof and some medical preparations thereof are disclosed in the published International Patent Application WO02/078694. The preparation is obtained by a chemical treatment of a chlorin-containing substance extracted from the biomass of Spirulina algae. It contains 80-90% of chlorin e6, 5-20% of purpurin 5, while chlorin p6 is the residue, i.e. practically it is a small admixture, if not an impurity, originating from the manufacturing process of the chlorin e6-based photosensitizer. The photosensitizer is also known under the name Radachlorin. The medical preparations described in this publication comprise a sterile 0.5% aqueous solution for injections (trade name Photochlorin), a solution in dimethylsulfoxide for external use, a gel for external use based on dimethylsulfoxide for external use, as well as gels on polymeric vehicles containing Pemulen, Carbopol or Auculyn. It has been described that the medical preparations can also contain variety of other additives, approved for pharmaceutical applications, such as calcium carbonate, sucrose, glucose, starch, magnesium stearate, various grades of polyvinylpyrrolidone, polyglucans, gel and emulsion vehicles, etc. However, no example of a preparation containing chlorin p6 alone has been provided.

Commercial preparation Fotolon for photodynamic therapy and diagnostics of cancers based on chlorin e6 photosensitizer (13-carboxy-17-[2-carboxyethyl]-15-carboxymethyl-17,18-trans-dihydro-3-vinyl-8-ethyl-2,7,12,18-tetramethylporphyrin) has obtained marketing authorizations in Russia and Belarus.

Chlorin p6 and its salts are relatively unstable in solutions at room temperature which causes serious difficulties in preparation and storage of medical preparations containing it.

The objective of this invention is to provide a novel photosensitizer of a high chemical and photochemical stability, good solubility both in water and in biological fluids, high affinity to the target region or the surface of the body or a tissue, large depth of necrosis, efficient generation of the active species that cause destruction of the pathologically changed tissue, low phototoxicity as well as to provide a method of preparation of such photosensitizer.

This objective has been achieved by providing a novel photosensitizing agent which is an association of polyvinylpyrrolidone with chlorin p6. Without being bound to theoretical considerations, the inventors have found and believe that chlorine p6 and its salts with alkali metals and its ammonium salt form a molecular complex with polyvinylpyrrolidone. The complex has all the aforementioned properties, in particular a high stability, also in a solution, without loosing efficacy of chlorin p6 as a photosensitizer. The association of the invention ensures also a deep penetration of irradiation into biological tissues, a high degree of pathological tissues necrosis and strong inhibition of neoplasm growth and therefore it increases also the average survival time.

### DESCRIPTION OF THE FIGURES OF THE DRAWING:

FIG. 1 shows the absorption spectra of the association of chlorin p6 with polyvinylpyrrolidone in the weight ratio of 1:1, 1:15, 1:40, 1:200 and 1:1,000;

FIG. 2 shows a shift of the maximum of light absorption in the long-wave region of spectrum for the association of chlorin p6 sodium salt with polyvinylpyrrolidone at various weight ratios-in comparison with chlorin p6 and commercial preparation Fotolon;

FIG. 3 shows the inhibition of the tumor growth following the photodynamic therapy using the association according to the invention (1:15, by weight) as a photosensitizer compared to that of chlorin p6 alone, the preparation Fotolon and the control.

FIG. 4 shows the “dose-response” relationship for the association according to the invention (1:15, by weight) in M-1 sarcoma model.

The object of the invention is an association of polyvinylpyrrolidone with chlorin p6 and/or its salt with a pharmaceutically acceptable counter-ion.

Polyvinylpyrrolidone used for the preparation of the association should be a linear, water-soluble grade of the polymer.

Preferably, molecular weight of polyvinylpyrrolidone contained in the association according to the invention is within the range of 5-20 kDa, more preferably within 5-15 kDa, in particular about 12.5 kDa.

Preferably, the weight ratio of chlorin p6 to polyvinylpyrrolidone in the association according to the invention is in the range from about 1:1 to about 1:40, more preferably from about 1:1 to about 1:15, in particular about 1:15.

Preferably, the pharmaceutically acceptable counter-ion in the association according to the invention is an alkali metal ion; in particular the ion selected from a group comprising sodium and potassium ions.

Another embodiment of the association according to the invention is the association of polyvinylpyrrolidone with chlorin p6 ammonium salt.

The association as defined above can be used as a photosensitizer, in particular as a photosensitizer in photodynamic therapy and/or diagnostics or for photochemical internalization of molecules.

The association as defined above is also useful as a photosensitizer in cosmetology.

The uses of the association according to the invention in cosmetology can include, for example, the use for hair depilation or removal of tattoos, naevi or skin hyperpigmentation.

The invention provides also a process for the preparation of the association as defined above, which comprises the following steps:
a) dissolving chlorin p6 in an aqueous solution of pH 8-13, preferably 12-13, containing suitable pharmaceutically acceptable counter-ion to yield a first solution;
b) dissolving polyvinylpyrrolidone in water or in an aqueous solution containing suitable pharmaceutically acceptable counter-ion, preferably at pH within the range 9.5-11.0 to yield a second solution;
c) mixing the first solution with the second solution;
d) optionally adjusting pH of the mixture to 7.5-9.0;
e) optionally sterilizing the mixture by filtration; and
f) subsequent lyophilization.

The association according to the invention can be used in the form of a pharmaceutical composition or a medical preparation.

Thus, the invention provides also a pharmaceutical composition containing the association of polyvinylpyrrolidone with chlorin p6 and/or with its salt with a pharmaceutically acceptable counter-ion and a pharmaceutically acceptable carrier and/or excipients.

The pharmaceutical composition according to the invention may be in a form of a solid, e.g. lyophilized preparation, for reconstitution prior to use in a liquid aqueous carrier for injections.

The pharmaceutical composition according to the invention may also be in a form of a preparation for external use, for instance in the form of a solution or a lotion in appropriate solvent, or an ointment, gel or cream in a typical vehicle, to be applied on the skin, in particular in cosmetology.

Furthermore, the pharmaceutical composition according to the invention may contain at least one additional active ingredient, such as an active ingredient effective in treatment of neoplastic diseases.

The object of the invention is also the use of the association according to the invention for the preparation of a medicament for use in photodynamic therapy and/or diagnostics or in photochemical internalization of molecules.

In one of the embodiments of the invention, the use relates to the preparation of a medicament for photodynamic therapy and diagnostics of neoplastic diseases as well as pre-neoplastic conditions.

The neoplastic diseases are, for instance, lung cancer, endobronchial lung cancer, urinary bladder cancer, esophagus cancer, skin cancer, breast cancer, brain tumor, colorectal cancer, gastric cancer, leucoplakia and gynecologic malignancies, such as tumors of the vagina, vulva and cervix.

In another embodiment of the invention, the use relates to the preparation of a medicament for photodynamic therapy of skin and mucous membrane diseases.

The skin disease is, for example psoriasis, radiation-induced skin keratosis, skin injuries including wounds, in particular purulent wounds, and erosions.

In still another embodiment of the invention, the use relates to the preparation of a medicament for photodynamic therapy of ophthalmological diseases, including senile macular degeneration, in particular the exudative form of age-related macular degeneration and subfoveal choroidal neovascularization in pathologic myopia, and post-inflammatory lessions.

The subject of the invention is also the method of photodynamic treatment and/or diagnostics of diseases and abnormalities, which comprises:
administration of the association or the pharmaceutical composition as defined above to a patient affected with a disease or abnormality; and then
exposure of the pathologically changed body region to the light of an appropriate wavelength, preferably in the range of 650-670 nm, especially 660-670 nm.

The object of the invention is also a method of cosmetic tretament of the human skin, comprising application the association or the pharmaceutical composition as defined above on the skin of a human subject in need of such treatment; and then
exposure of the region of the skin to the light of an appropriate wavelength, preferably in the range of 650-670 nm, especially 660-670 nm.

The invention is further illustrated by the following examples, which do not limit its scope.

### EXAMPLE 1

Preparation of the 1:1 Association

1.0 g of chlorin p6 was dissolved with stirring in 0.6 l of an aqueous solution of sodium hydroxide (pH 12.4). The thus-obtained first solution was stirred for 35 min. The second solution was prepared by dissolving 1.0 g of polyvinylpyrrolidone (average molecular weight 12,600 Da) in 0.1 l of an aqueous solution of sodium hydroxide (pH 10.5). The first and second solutions were combined and stirred for 30 min. and then pH of the resulting mixture was adjusted to 8.1. The association (complex) of chlorin p6 with polyvinylpyrrolidone was obtained as an aqueous solution. The solution was then sterilized by filtration, frozen and lyophilized to yield the ready to use association of polyvinylpyrrolidone with chlorin p6 (1:1 by weight).

### EXAMPLE 2

Preparation of the 1:15 Association

1.0 g of chlorin p6 was dissolved with stirring in 0.5 l of an aqueous solution of sodium hydroxide (pH 12.7). The thus-obtained first solution was stirred for 35 min. The second solution was prepared by dissolving 15.0 g of polyvinylpyrrolidone (average molecular weight 12,600 Da) in 0.31 of an aqueous solution of sodium hydroxide (pH 10.9). The first and second solutions were combined, pH of the resulting solution was adjusted to 8.4 and then the mixture was stirred for 35 min. The association (complex) of chlorin p6 with polyvinylpyrrolidone was obtained as an aqueous solution. The solution was then sterilized by filtration, frozen and lyophilized to yield the ready to use association of chlorin p6 with polyvinylpyrrolidone (1:15 by weight).

### EXAMPLE 3

Preparation of the 1:200 Association

1.0 g of chlorin p6 was dissolved with stirring in 0.5 l of an aqueous solution of sodium hydroxide (pH 12.8). The thus-obtained first solution was stirred for 40 min. The second solution was prepared by dissolving 200.0 g of polyvinylpyrrolidone (average molecular weight 12,600 Da) in 0.5 l of an aqueous solution of sodium hydroxide (pH 10.9). The first and second solutions were combined, pH of the resulting solution was adjusted to 8.2 and then the mixture was stirred for 40 min. The association of chlorin p6 with polyvinylpyrrolidone was obtained as an aqueous solution. The solution was then sterilized by filtration, frozen and lyophilized to yield the ready to use association of chlorin p6 with polyvinylpyrrolidone (1:200 by weight).

### EXAMPLE 4

Investigation of Stability of the Association According to the Invention

Stability of the association according to the invention (weight ratio chlorin p6:polyvinylpyrrolidone=1:15) compared to that of the commercially-available photosensitizer Fotolon was investigated using a stress method. The content of active ingredient in the association according to the invention was 97.4%, that in the Fotolon—87.3%.

a) Investigation of Thermal Stability

Samples of the aforementioned preparations were heated at 60° C. in an open flask in a climatic chamber protected against light. 10 mg of a powdered sample was placed as a thin layer in a 20 ml flask. The content of the active ingredients in the tested photosensitizers was determined by liquid chromatography after 7 and 14 days of the test. The results are shown in Table 1.

TABLE 1
Changes of the active ingredients content in samples of the
association according to the invention and of Fotolon
in the stress study (heating at 60° C.).

| | Content of the active ingredient | | |
|---|---|---|---|
| | with respect to the initial value, % | | |
| Sample | Initial value | 7 days | 14 days |
| Association according | 100 | 99.8 | 99.9 |
| to the invention | | | |
| Fotolon | 100 | 92.9 | 91.9 |

b) Oxidative Stress

2 mg/ml solutions of the above-defined samples in aqueous hydrogen peroxide (3%) were prepared. The solutions were kept at 22° C. without exposure to light. The content of the active ingredients in tested photosensitizers was determined after 2, 7 and 14 days of the test. The results are shown in Table 2.

TABLE 2
Changes of the active ingredients content in samples of the
association according to the invention and of Fotolon
in the stress study (oxidative stress).

| | Content of the active ingredient | | | |
|---|---|---|---|---|
| | with respect to the initial value, % | | | |
| Sample | Initial value | 2 days | 7 days | 14 days |
| association according | 100 | 94.9 | 84.9 | 71.6 |
| to the invention | | | | |
| Fotolon | 100 | 6.0 | <5.0 | <5.0 |

c) Resistance to Hydrolysis in a Solution

Stability of the association according to the invention and Fotolon was tested in a solution (pH=7.0). A 0.05M phosphate buffer was used for preparing the tested samples. The samples were titrated until pH of the solution was equal to 7.0. Concentration of the photosensitizer in the prepared solutions was 2 mg/ml. Flasks containing the solutions were capped with plugs and kept at 22° C. in a dark place. Assay of the active ingredient in the tested photosensitizers was performed for 14 days. The results are shown in Table 3.

TABLE 3
Changes of the active ingredients content in samples of the
association according to the invention and Fotolon in the stress study
(resistance to hydrolysis in solutions at various pH values)

Content of the active ingredient
with respect to the initial value, %

| Sample | Initial value | pH 7.0 |
|---|---|---|
| association according to | 100 | 95.0 |
| the invention | | |
| Fotolon | 100 | 65.6 |

The results of stability tests of the photosensitizers under stress conditions, shown in Tables 1, 2 and 3 show that the association according to the invention is more stable than Fotolon.

### EXAMPLE 5

Investigation of Light Absorption by the Association According to the Invention

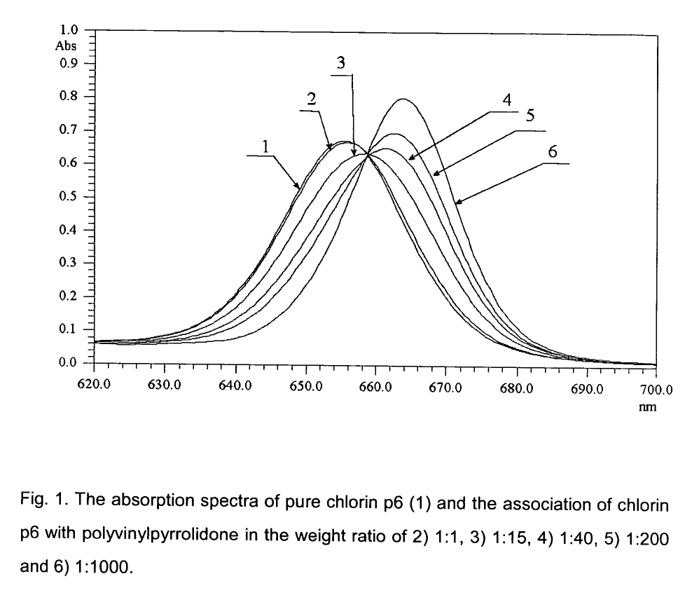

The absorption spectra of the above-obtained associations of polyvinylpyrrolidone with chlorin p6 in the phosphate buffer (0.05M, pH 8.5) are shown on FIG. 1 for the following chlorin p6/polyvinylpyrrolidone ratios (by weight): (1) 1:1; (2) 1:15; (3) 1:40; (4) 1:200; and (5) 1:1000. The concentration of chlorin p6 was 1.9·10⁻⁵M in all investigated solutions.

The chlorin p6-polyvinylpyrrolidone complex has an absorption maximum in the long-wave region of visible light, and this maximum is shifted towards longer wavelengths (664 nm) compared to free chlorin p6, which has an absorption maximum at 656 nm. Therefore, the use of the association according to the invention as a photosensitizer allows for deeper penetration of radiation into biological tissues, which allows for treating solid tumors and/or deeply located lesions.

Formation of the complex of polyvinylpyrrolidone with chlorin p6 results in a series of changes in the absorption spectrum. Along with increasing weight share of chlorin p6 in the complex, a bathochromic shift of the long-wave absorption maximum is observed from 656 nm for free chlorin p6 to 664 nm for the complex. Simultaneously, several isosbestic points are observed in the absorption spectrum, which evidence the presence of two equivalent states of chlorin p6, i.e., a transformation from free chlorin p6 to its complex. The formation constant of the chlorin p6-polyvinylpyrrolidone complex having MW of 12 kDa was found to be 1.51·10⁶ M⁻¹.

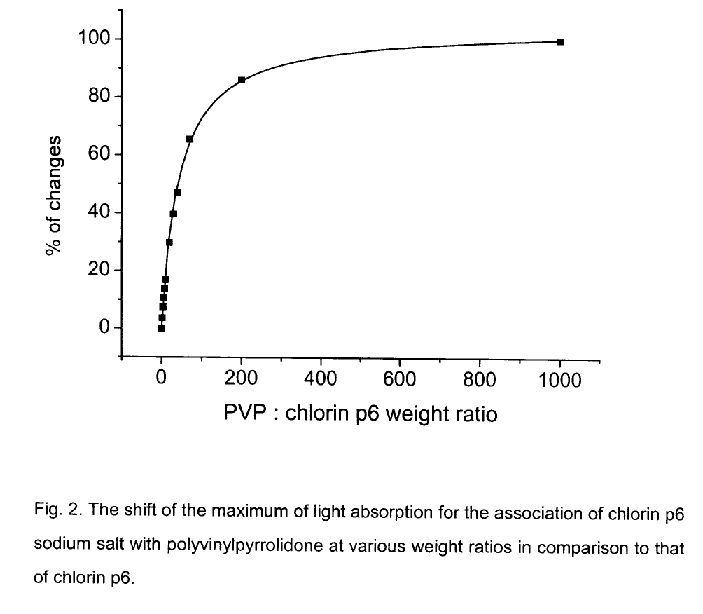

FIG. 2 presents the experimentally obtained percent of red shift of the absorption maximum of the complex in relation to the content of polyvinylpyrrolidone. These results can be used to determine the lower and upper limit of the weight ratio of chlorin p6 to polyvinylpyrrolidone. The spectra were recorded for a series of the solutions of the complex in the phosphate buffer (pH 8.5) at the chlorin p6 concentration of 1.9·10⁻⁵M.

As it can be seen from the relationship shown in FIG. 2, the increment of red shift is practically equal to zero when the content of polyvinylpyrrolidone in the complex is higher than 200 weight parts per one part of chlorin p6. Besides, the higher contents of polyvinylpyrrolidone in the association results in high viscosity of the solution, which creates problems concerning its administration and might cause adverse reactions.

If the content of polyvinylpyrrolidone in the association is lower than the weight fraction of chlorin p6 (<1), there are no significant differences between the spectral properties of chlorin p6 and thoseof the association with polyvinylpyrrolidone.

### EXAMPLE 6

Investigation of the Yield of the Cytotoxic Species

Spectral and energetic characteristics of the association according to the invention were investigated in order to determine its effectiveness in generating active species. The parameters that quantitatively describe the process are duration of the triplet state (S_{T}) and the quantum yield of singlet-state oxygen (φλ). Fotolon was used as a reference.

The relative quantum yield of the singlet-state oxygen was determined by measuring integral fluorescence intensity of solutions of the tested samples in ethanol. A solution of tetrasodium phenylporphyrintetrasulfonate (TSFP, φλ=0.7).

TABLE 4
Photosensitization characteristics of the association according
to the invention (1:15) and Fotolon in ethanolic solutions

| | Duration of the | Quantum yield of the |
|---|---|---|
| Sample | triplet state | singlet-state oxygen |
| Association according | 263 ± 3 | 0.71 ± 0.07 |
| to the invention | | |
| Fotolon | 255 ± 3 | 0.60 ± 0.06 |

The results of the studies show that duration of the triplet state is sufficient for an effective diffusive collision of excited molecules of the photosensitizer and oxygen that results in formation of singlet-state oxygen. The quantum yield of singlet-state oxygen is higher for the association according to the invention which proves a more efficient generation of cytotoxic species that is a prerequisite of higher photodynamic efficacy.

### EXAMPLE 7

Efficacy of the Photodynamic Therapy

The results of studies on tumor necrosis by photodynamic therapy also confirm that the use of the association according to the invention allows to obtain a much higher efficacy of the therapy due to increased tumor necrosis with respect to that attainable by the use of chlorin p6 alone or the commercially available preparation Fotolon.

The studies were performed on white not-inbred laboratory rats having subcutaneously implanted tumors (Pliss' lymphosarcoma). The dose of the administered photosensitizer was 2.5 mg/kg. The necrosis area was estimated based on the functional condition of the tumor's vascular system following laser irradiation (660 nm) at the radiation dose of 50 J/cm², when accumulation of the photosensitizer was the highest.

After 24 hours since the photodynamic therapy, the animals were intravenously given a solution of Evans blue in physiologic saline at the dose of 1.0 ml/100 g of body weight.

The experiments have shown that in the Pliss' lymphosarcoma model the necrosis area is 46.3% for chlorin p6, 61% for Fotolon and 89.4% for the photosensitizer based on the association according to the invention (1:15)—89.4%. The depth of necrosis was 7, 16 and 22 mm, respectively.

Determinations of the necrosis depth associated with the use of the association according to the invention were performed also on the M-1 sarcoma and RS-1 follicular liver cancer models.

The maximal depth of necrosis was noted for the RS-1 follicular liver cancer model. It has reached 32 mm, which corresponded to the maximal linear size of the neoplasm. No studies were performed on larger size neoplasms.

The experimental data are presented in Tables 5 and 6.

TABLE 5
The depth of necrosis in the model of subcutaneously implanted
M-1 sarcoma. Dose of the photosensitizer according to the invention:
5 mg/kg; dose of irradiation: 50 J/cm³.

| | | | |
|---|---|---|---|
| Number of the tumor section | 1 | 2 | 3 |
| Depth of tumor necrosis, mm | 14 | 10 | 11 |
| Linear size of the tumor section, mm | 14 | 10 | 11 |

TABLE 6
The depth of necrosis in the model of RS-1 follicular liver cancer.
Dose of the photosensitizer according to the invention: 2.5 mg/kg;
dose of irradiation: 100 J/cm³.

| | | | | | | | | |
|---|---|---|---|---|---|---|---|---|
| Number of the tumor section | 1 | 2 | 3 | 4 | 5 | 6 | 7 | 8 |
| Depth of tumor necrosis, mm | 32 | 27 | 26 | 23 | 25.6 | 30 | 30 | 30 |
| Linear size of the tumor section, | 32 | 27 | 28 | 23 | 25.6 | 30 | 30 | 30 |
| mm | | | | | | | | |

Thus, the results of the tests show that the depth of tumor necrosis following the photodynamic therapy with the use of the association according to the invention is up to 32 mm.

One of the most important characteristics of the anti-neoplastic therapy that determines its therapeutic efficacy is the tumor growth inhibition factor.

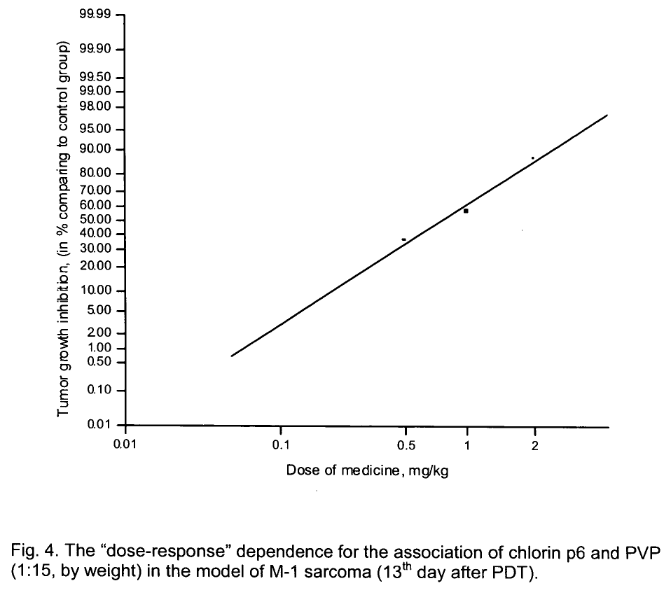

Investigations of the activity of the photosensitizer—the association according to the invention—in inhibiting the tumor growth were performed on rats using subcutaneously implanted M-1 sarcoma as a model. In the experiment, the photosensitizer was administered in three doses: 0.5, 1.0 and 2.0 mg/kg. Each dose was given to a group of rats consisting of 6 animals. The control group consisted also of 6 rats that were not treated. The animals in each of the tested groups were given appropriate doses of the preparation and after 4 hours they were irradiated by a laser light of the wavelength 663 nm at a dose of 50 J/cm². Evaluation of the tumor growth inhibition (TGI) factor, expressed in percents, was performed after 13 days from the start of the photodynamic therapy (FIG. 4). FIG. 4 illustrates TGI factor as a function of the dose of the photosensitizer.

On the basis of the obtained experimental data it was concluded that the association according to the invention has statistically significant effect of inhibiting growth of M-1 sarcoma.

At the dose of about 0.9 mg/kg 50% inhibition of the tumor growth was reached. At the dose of 2 mg/kg inhibition of the tumor growth reached 80-90% within 13 days.

By extrapolating the data it can be determined that at given dose of irradiation (50 J/cm²), 100% of TGI could be achieved at the dose of the photosensitizer of approximately 2.4 mg/kg.

Thus, the association according to the invention allows to achievie 100% TGI even at relatively low doses of the preparation (2.4 mg/kg) and irradiation (50 J/cm²).

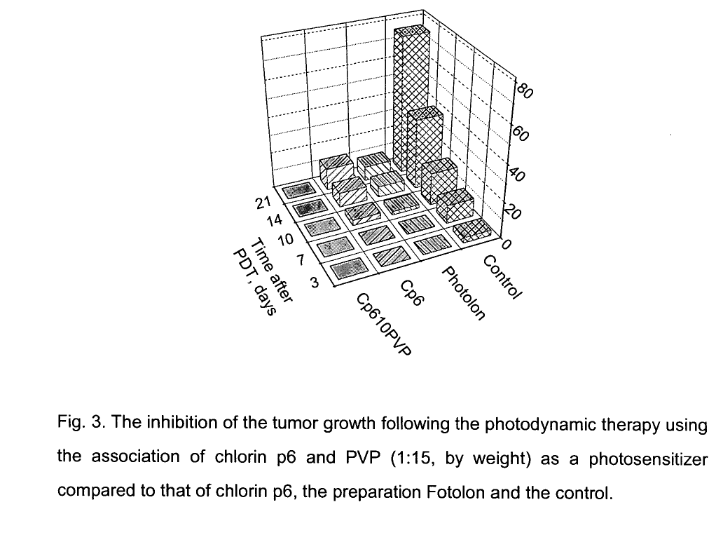

The use of the association according to the invention allows to achieve a high level of the tumor growth inhibition. In order to confirm this effect, the studies were performed using white rats with implanted neoplasms (Walker-256 carcinosarcoma). A solution of the photosensitizer was administered intraperitoneally at doses of 2.5 mg/kg and the tumor was irradiated for 5 hours at the radiation dose of 200 J/cm². The observation was continued for 21 days following the therapy. FIG. 3 and Table 7 show the dynamics of changes of the absolute rate of the tumor growth, resulting from the use of the association according to the invention (1:15) as well as free chlorin p6. The control group consisted of untreated animals.

TABLE 7
Inhibition of neoplasm's growth

| | Neoplasm's growth, % | | | |
|---|---|---|---|---|
| Time elapsed | association | | | |
| after photodynamic | according to | | | |
| therapy, days | the invention (1:15) | Chlorin p6 | Fotolon | Control |
| 3 | 0 | 0 | 0 | 3 |
| 7 | 0 | 0 | 0 | 10 |
| 10 | 0 | 3 | 2 | 18 |
| 14 | 0 | 8 | 6 | 39 |
| 21 | 0 | 12 | 8 | 76 |

As it results from the experimental data, no tumor growth is observed within 21 days following the use of the association according to the invention as a photosensitizer. In the case of using chlorin p6 alone or Fotolon, the tumor growth resumes after 10 days following the therapy.

One of the most important characteristics of anti-neoplastic therapies is the increase of survival time.

Considering the results obtained in studies on inhibition of the tumor growth at various doses of the photosensitizer (from 0.5 to 2.5 mg/kg), a dose of the association according to the invention equal to 2.5 mg/kg and a dose of irradiation equal to 100 J/cm² were chosen for the experiment aimed at determination of the average survival time (AST) of rats with implanted M-1 sarcoma. As in the above described example, the photosensitizer was administered intravenously and irradiation was performed after 4 hours.

In the experiment it was found that for the control group of animals (without treatment) AST was 33.7 days, while in the tested group (M-1 sarcoma) it reached 81.8 days.

On the basis of the above data, the survival time index (STI) for the animals can be calculated as follows:

$$STI = \frac{{AST}_{test} - {AST}_{contr}}{{AST}_{contr}} \times 100 \% = 142.7 \%$$

and if the T/C (treated/control) ratio is to be used:

$$T / C = \frac{{AST}_{test}}{{AST}_{contr}} \times 100 \% = 242.7 \%$$

If the T/C factor is within the range 201-300% at a single dose of a preparation, the preparation is considered as highly efficacious (+++).

Thus, the obtained data allow to describe the association according to the invention as a highly efficacious preparation.

It was thus demonstrated that the association according to the invention has advantageous features compared to free chlorin p6 and the known commercially available preparation Fotolon. Its higher anti-neoplastic activity manifests in the more extensive tissue penetration, necrosis, and inhibition of the tumor growth following the photodynamic therapy as well as in significantly increased average survival time.

### EXAMPLE 8

Photodynamic Therapy of Ophthalmologic Diseases

Efficacy of the association according to the invention in the photodynamic therapy of ophthalmological diseases was studied on the rabbit model of ocular neovascularization induced by laser coagulation. The studies were conducted on a group of 10 animals. The association according to the invention (1:15) at a concentration 2 mg/l was given intravenously to the animals at a dose of 6-8 mg/m² of body surface. After 5 minutes following administration of the photosensitizer, irradiation of the choroidal neovascularization zones by laser light at 600-700 mW/cm² was performed over 83 seconds. The extent of damage of the neovascularizing membrane was estimated as a measure of efficacy of the photodynamic therapy.

The studies have demonstrated that the association according to the invention provides 100% occlusion of the vasculature of the model choroidal neovascularization and inhibits neoangiogenesis processes.

### EXAMPLE 9

The Use of the Association According to the Invention for Treating Purulent Wounds and Erosions

The studies were performed on rats which had full, flat wounds having a surface area of 375mm², infected with Staphylococcus spp. The thus-obtained purulent wounds were washed with 1.0 mg/ml solution of the association according to the invention (1:15) and then irradiated by laser light at 5 mWatt over 5-10 minutes. Efficacy of therapy was evaluated by observations of wound healing and a general condition of the animals.

The studies have demonstrated that the use of the association according to the invention accelerates the restorative processes, clearing the wounds from pus, tearing away the original crust and developing the granular tissue.

Therefore, the association according to the invention has a high photodynamic activity in treating malignant tumors, manifested in high extent of necrosis following the photodynamic therapy and provides a deep penetration of the radiation into the pathologic tissue.

1. An association of polyvinylpyrrolidone with chlorin p6 and/or its salt with a pharmaceutically acceptable counter-ion.

2. The association according to claim 1, wherein the weight ratio of chlorin p6 and/or its salt with a pharmaceutically acceptable counter-ion to polyvinylpyrrolidone is within the range from about 1:1 to about 1:200.

3. The association according to claim 2, wherein the weight ratio of chlorin p6 and/or its salt with a pharmaceutically acceptable counter-ion to polyvinylpyrrolidone is within the range from about 1:1 to about 1:40.

4. The association according to claim 3, wherein the weight ratio of chlorin p6 and/or its salt with a pharmaceutically acceptable counter-ion to polyvinylpyrrolidone is within the range from about 1:1 to about 1:20.

5. The association according to claim 4, wherein the weight ratio of chlorin p6 and/or its salt with a pharmaceutically acceptable counter-ion to polyvinylpyrrolidone is within the range from about 1:10 to about 1:20.

6. The association according to claim 4, wherein the weight ratio of chlorin p6 and/or its salt with a pharmaceutically acceptable counter-ion to polyvinylpyrrolidone is approximately 1:15.

7. The association according to claim 1 wherein the molecular weight of polyvinylpyrrolidone is within the range 5-20 kDa.

8. The association according to claim 7 wherein the molecular weight of polyvinylpyrrolidone is within the range 5-15 kDa.

9. The association according to claim 8 wherein the molecular weight of polyvinylpyrrolidone is approximately 12.5 kDa.

10. The association according to claim 1, wherein the pharmaceutically acceptable counter-ion is an alkali metal ion.

11. The association according to claim 10, wherein the alkali metal ion is selected from the group consisting of sodium and potassium ions.

12. The association according to claim 1, wherein the pharmaceutically acceptable counter-ion is ammonium ion.

13. The association according to claim 1, wherein the pharmaceutically acceptable counter-ion is sodium ion, the weight ratio of sodium salt to polyvinylpyrrolidone is approximately 1:15 and molecular weight of polyvinylpyrrolidone is approximately 12.5 kDa.

14. The association according to claim 1, wherein chlorin p6 and/or its salt forms a molecular complex with polyvinylpyrrolidone.

15. The association according to claim 1, for use as a photosensitizer.

16. The association according to claim 15 for use as a photosensitizer in photodynamic therapy and/or diagnostics or for photochemical internalization of molecules.

17. The association according to claim 15 for use as a photosensitizer in cosmetology.

18. The association according to claim 17 for use in hair depilation or tattoo removal.

19. A pharmaceutical composition containing an association of polyvinylpyrrolidone with chlorin p6 and/or its salt with a pharmaceutically acceptable counter-ion and a pharmaceutically acceptable carrier and/or excipients.

20. The pharmaceutical composition according to claim 19 containing at least one additional active ingredient.

21. The pharmaceutical composition according to claim 20 wherein the additional active ingredient is effective in the treatment of neoplastic diseases.

22. A process for the preparation of the association as defined in claim 1, comprising the following steps:
a) dissolving chlorin p6 in an aqueous solution of pH 8-13 containing suitable appropriate pharmacologically acceptable counter-ion to obtain a first solution;
b) dissolving polyvinylpyrrolidone in water or in an aqueous solution containing an appropriate pharmaceutically acceptable counter-ion to obtain a second solution;
c) mixing the first and the second solutions;
d) optionally adjusting the pH value of the mixture to 7.5-9.0;
e) optionally sterilizing the mixture by filtration; and
f) subsequent lyophilization.

23. The process according to claim 22 wherein the pH value in the step a) is within the range 12-13.

24. The process according to claim 22 wherein the pH value in the step b) is within the range 9.5-11.0.

25-30. (canceled)

31. A method of photodynamic treatment and/or diagnosis of diseases and abnormalities, comprising:
a) administration of the composition defined as in claim 19 to a patient affected with a disease or abnormality; and
b) exposure of a pathologically changed region of the body to the light of an appropriate wavelength.

32. The method according to claim 31 comprising exposure to the light of a wavelength within the range 650-670 nm.

33. The method according to claim 31 wherein the disease is a neoplastic disease or a pre-neoplastic condition.

34. The method according to claim 32 wherein the neoplastic disease is selected from the group consisting of lung cancer, endobronchial lung cancer, urinary bladder cancer, esophagus cancer, skin cancer, breast cancer, brain tumor, colorectal cancer, gastric cancer, leucoplakia and gynecologic malignancies, such as tumors of the vagina, vulva and cervix.

35. The method according to claim 31 wherein the disease is a skin or mucous membrane disease.

36. The method according to claim 34 wherein the skin disease is psoriasis, radiation-induced skin keratosis, skin injuries, including wounds, purulent wounds and erosions.

37. The method according to claim 31 wherein the disease is an ophthalmic disease, including age-related macular degeneration, the exudative form of age-related macular degeneration and subfoveal choroidal neovascularization in pathologic myopia, and post-inflammatory lessions.

38. A method of cosmetic treatment of the human skin comprising the application of the composition defined as in claim 19 on the area of the skin of the human subject in need of such treatment and then exposure of said region of the skin to the light of an appropriate wavelength.

39. The method according to claim 38 wherein the cosmetic treatment is hair depilation or tattoo removal.

40. The method according to claim 38 involving exposure to the light of a wavelength within the range 650-670 nm.

41. The method according to claim 38 involving exposure to the light of a wavelength within the range 660-670 nm.

42. The method according to claim 31 comprising exposure to the light of a wavelength within the range 660-670 nm.
